(19) United States
(12) Patent Application Publication
(10) Pub. No.: US 2009/0236284 A1
(43) Pub. Date: Sep. 24, 2009

(54) REMOVAL OF SUBSTANCES IN DIALYSIS SOLUTIONS AND DIALYSIS COMPONENTS BY ION EXCHANGE ADSORPTION

(75) Inventors: Richard Johnson, Mundelein, IL (US); Beverly Johnson, Mundelein, IL (US); Valerie Leesch, Buffalo Grove, IL (US)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/052,508

(22) Filed: Mar. 20, 2008

(51) Int. Cl. B01D 15/04

(52) U.S. Cl. 210/638

(57) ABSTRACT

Methods for removing substances from dialysis solutions and dialysis components using ion exchange adsorption are provided. In a general embodiment, the present disclosure provides a method for removing a substance from a dialysis solution. The method comprises providing a dialysis solution and passing the dialysis solution through an ion exchange resin so that at least a portion of the overall amount of the substance is removed from the dialysis solution by being adsorbed onto the ion exchange resin. The ion exchange resin can comprise an anion exchange resin or a cation exchange resin depending on the substances that are desired to be removed from the dialysis solution.

### BACKGROUND

The present disclosure relates generally to dialysis solutions. More particularly, the present disclosure relates to methods for removing substances from dialysis solutions or dialysis components using ion exchange adsorption.

Parenteral pharmaceutical products are required to be free of contaminating substances, such as those that might cause peritonitis. Peritonitis, or inflammation of the peritoneum, is a major complication of peritoneal dialysis. Peritonitis may be caused by intraperitoneal bacterial infections. Alternatively, peritonitis caused by a chemical or a foreign body irritant is known as aseptic or sterile peritonitis. Despite existing testing of peritoneal dialysis solutions, outbreaks of aseptic peritonitis still occur.

### SUMMARY

The present disclosure generally relates to methods for removing substances from dialysis solutions or dialysis components (i.e. raw materials used to manufacture dialysis solutions). In a general embodiment, the present disclosure provides a method for removing a substance from a dialysis solution (e.g. peritoneal dialysis solution) or dialysis components used to make a dialysis solution using ion exchange adsorption. The method comprises providing a dialysis solution and passing the dialysis solution through one or more ion exchange resins so that at least a portion of the overall amount of the substance is removed from the dialysis solution by being adsorbed onto the ion exchange resin. The ion exchange resin can comprise an anion exchange resin or a cation exchange resin depending on the ionic charges of the substances that are desired to be removed from the dialysis solution.

In an embodiment, the method further comprises spray drying the dialysis solution to form a spray dried dialysis ingredient after the dialysis solution has passed through the ion exchange resin. The spray dried dialysis ingredient can then be packaged into a sterile container. In an alternate embodiment, the method further comprises packaging the dialysis solution into a sterile container after the dialysis solution has passed through the ion exchange resin.

In an embodiment, the method comprises passing a basic solution through the ion exchange resin prior to passing the dialysis solution through the ion exchange resin. This prepares the ion exchange sites on the ion exchange resin to remove the substance(s) from the dialysis solution. The basic solution can comprise a pH greater than 7.0.

In another embodiment, the present disclosure provides a method for removing a substance from one or more peritoneal dialysis components such as glucose polymers or glucose polymer derivatives. For example, the substance can be a microbial contaminant. The peritoneal dialysis components can be dissolved in a solution such as sterile water and the dissolved peritoneal dialysis components solution run over an ion exchange column or run through a membrane modified with cation or anion exchange groups to remove any ionic microbial components that may be in the peritoneal dialysis components at low levels.

In yet another embodiment, the present disclosure provides methods for manufacturing a peritoneal dialysis solution. The method can include any suitable number and type of processing stages. For example, the method comprises providing at least one peritoneal dialysis component, dissolving the dialysis component in a solution and passing the dialysis component solution through an ion exchange resin so that at least a portion of the overall amount of the charged contaminant is removed from the solution by being adsorbed onto the ion exchange resin. After the contaminant is removed, the dialysis component solution can transformed back into a dried dialysis component (e.g. via spray drying). The dried dialysis component can then be used in preparing the dialysis solution.

In another embodiment, the present disclosure provides a method for removing microbial contaminants in a peritoneal dialysis solution. The method comprises providing a peritoneal dialysis solution and passing the dialysis solution through one or more anion exchange resins so that at least a portion of the overall amount of the microbial contaminants are removed from the dialysis solution by being adsorbed onto the ion exchange resin. The microbial contaminants that are removed from the dialysis solution can be peptide, glycan, lipoteichoic acid or combinations thereof.

In an alternative embodiment, the present disclosure provides a method of providing dialysis to a patient. The method comprises providing a dialysis solution, passing the dialysis solution through one or more ion exchange resins so that the substances are removed from the dialysis solution by being adsorbed onto the ion exchange resin and administering the dialysis solution to the patient.

An advantage of the present disclosure is to provide improved methods for removing a substance from dialysis solutions and/or dialysis components.

Another advantage of the present disclosure is to provide improved methods for manufacturing dialysis solutions or components used to make dialysis solutions.

Yet another advantage of the present disclosure is to provide improved dialysis solutions.

Still another advantage of the present disclosure is to provide improved safety procedures that can be employed to prevent peritonitis in patients that receive peritoneal dialysis therapy.

Another advantage is of the present disclosure is to provide improved methods for administering dialysis solutions to a patient.

Additional features and advantages are described herein, and will be apparent from the following Detailed Description and the figures.

### BRIEF DESCRIPTION OF THE FIGURES

FIG. 1 is a graph showing the IL-6 responses of a control dialysis solution and an implicated dialysis solution before and after exposure of the solutions to a DEAE SEPHAROSE® column, Run #1.

FIG. 2 is a graph showing mass balance results for icodextrin in DEAE SEPHAROSE® fractions, Run #2.

FIG. 3 is a graph showing the IL-6 responses of control and implicated dialysis solutions before and after exposure to a DEAE SEPHAROSE® column, Run #3, where the column and dialysis solutions were scaled up.

### DETAILED DESCRIPTION

The present disclosure generally relates to methods of removing substances from dialysis solutions or dialysis components (i.e. raw materials used to manufacture dialysis solutions). For example, the substances can be unwanted microbial contaminants that may be found in the pharmaceutical solutions such as dialysis solutions or dialysis components. Current dialysis solution manufacturing processes may permit small levels of microbial contaminants to enter the final formulation and risk creating adverse responses in patients. Incorporation of a capture or removal step that ensures these potential microbial contaminants are removed prior to formulation and filling would prevent this outcome.

Specific microbial contaminants that may be found in dialysis solutions can be, for example, pro-inflammatory substances such as peptidoglycan. Peptidoglycan is a major component of a gram positive bacterial cell wall and thus can serve as a marker for gram positive bacteria. In this regard, removing microbial substances such as peptidoglycan from dialysis solutions can be utilized to effectively prevent peritonitis in patients that use the dialysis solutions. Examples of dialysis solutions include peritoneal dialysis solutions that contain a glucose polymer such as icodextrin and the like.

Icodextrin is derived from corn starch, a natural product. It is well known that products of natural origin are contaminated with a wide variety of micro-organisms. The inventors have found that some natural products, such as corn starch, contain an acidophilic thermophilic bacteria, such as Alicyclobacillus acidocaldarius. The later organism is ubiquitous in the food industry, particularly in acidic beverages. It is the alicyclobacillus that produces guaiacol, which is a causative substance for an “off” flavor orange juice.

Aseptic peritonitis associated with icodextrin-based peritoneal dialysis solutions is believed to be the largest adverse event reported for a peritoneal dialysis solution due to a contaminant of microbial origin. Based on the experimental investigations, peptidoglycan in the glucose polymer or glucose polymer derivative-based peritoneal dialysis solution may be a causative agent of aseptic peritonitis. Further, pharmacovigilence data from previous studies supports the effectiveness of a corrective action and manufacturing screening procedure to prevent the occurrence of peritonitis. These findings illustrate that while endotoxin is deservedly one of the more worrisome bacterial product that can cause adverse effects to patients, it is not the sole one. In this regard, non-endotoxin pyrogens, such as peptidoglycans, are capable of producing clinically significant inflammation. Thus, this demonstrates that parenteral pharmaceutical products that pass the compendial tests and so meet Pharmacopoeia standards may still require a further level of testing to effectively determine the efficacy and safe use of such products to better ensure quality of life issues associated with use of same.

In a general embodiment, the present disclosure provides a method for removing a substance from a dialysis solution such as a peritoneal dialysis solution. The method comprises providing a dialysis solution and passing the dialysis solution through one or more ion exchange resins so that at least a portion of the overall amount of the substance is removed from the dialysis solution by being adsorbed onto the ion exchange resin. The ion exchange resin can comprise an anion exchange resin or a cation exchange resin depending on the ionic charges of the substances that are desired to be removed from the dialysis solution.

In another embodiment, any suitable dialysis component such as a glucose polymer or a glucose polymer derivate could be dissolved in sterile water and the dissolved pharmaceutical compound solution/dialysis component solution run over an ion exchange column or run through a membrane modified with cation or anion exchange groups to remove any ionic microbial components that may be in the pharmaceutical compounds at low levels. The ion exchange resin matrix can be washed to remove the bound microbial contaminants, sterilized by various methods and reused multiple times to lower the cost of performing this polishing/removal step.

An ion exchange capture step can be incorporated into the processing and manufacturing of dialysis solutions/dialysis components to remove any potential low level substances such as microbial contaminants from the dialysis solution/dialysis components, for example, before final formulation and filling operations. This polishing step may reduce the risk of adverse responses in the final dialysis product. Moreover, the ion exchange capture step could be made cost effective by re-charging and re-using the ion exchange resin.

The ion exchange resins can be in the form of resin beads (or any other suitable shape) that are packed into an ion exchange column. The ion exchange resins can also be in the form of a membrane modified with cation or anion exchange groups attached to a suitable support. The pharmaceutical/dialysis solutions can then be passed through the ion exchange column or the membranes at any suitable flow rate. The ion exchange resins can also be used in combination with other high or low molecular weight filters that are capable of removing any undesirable and uncharged materials from the pharmaceutical solutions.

In an embodiment, the method further comprises spray drying the dialysis solution to form a spray dried dialysis ingredient after the dialysis solution/dialysis component solution has passed through the ion exchange resin. The spray dried dialysis ingredient can then be packaged into a sterile container. The spray dried dialysis ingredient can be reconstituted with a suitable solution at the time of the dialysis therapy. In an alternate embodiment, the method further comprises packaging the dialysis solution into a sterile container after the dialysis solution has passed through the ion exchange resin.

In an embodiment, the method comprises passing a basic solution through the ion exchange resin prior to passing the dialysis solution/dialysis components through the ion exchange resin. This prepares the ion exchange sites on the ion exchange resin to remove the substance(s) from the dialysis solution. The basic solution can comprise a pH greater than 7.0.

In yet another embodiment, the present disclosure provides methods for manufacturing a dialysis solution. The method can include any suitable number and type of processing stages. For example, the method comprises providing at least one dialysis component, dissolving the dialysis component in a solution and passing the dialysis component solution through an ion exchange resin so that at least a portion of the overall amount of the charged contaminant is removed from the solution by being adsorbed onto the ion exchange resin. After the contaminant is removed, the dialysis component solution can transformed back into a dried dialysis component (e.g. via spray drying). The dried dialysis component can then be used in preparing the dialysis solution.

The dialysis solutions can be specifically formulated and suitable for peritoneal dialysis or any other dialysis therapies. The dialysis solutions can be used, for example, as a single dialysis solution in a single container or as a dialysis part of a separately housed or multi-chambered container. The dialysis solutions can be sterilized using any suitable sterilizing technique such as, for example, autoclave, steam, ultra-violet, high pressure, filtration or combination thereof.

The method of manufacturing a dialysis solution in accordance with the present disclosure can also be used in conjunction with other suitable dialysis component or dialysis solution testing procedures. Illustrative examples of suitable testing procedures can be found in U.S. Pat. No. 7,118,857, entitled METHODS AND COMPOSITIONS FOR DETECTION OF MICROBIAL CONTAMINANTS IN PERITONEAL DIALYSIS SOLUTIONS, issued on Oct. 10, 2006, the disclosure of which is herein incorporated by reference. For example, such testing procedures can be generally used to test dialysis components or dialysis solutions for microbial contaminants. The dialysis solution or dialysis component can be further processed to remove the contaminant or to achieve a sufficiently low level of the contaminant in accordance with embodiments of the present disclosure.

In another embodiment, the present disclosure provides a method for removing microbial contaminants in a dialysis solution/solution of dialysis components. The method comprises providing a dialysis solution and passing the dialysis solution through one or more anion exchange resins so that at least a portion of the overall amount of the microbial contaminants are removed from the dialysis solution by being adsorbed onto the ion exchange resin. The microbial contaminants that are removed from the dialysis solution can be peptide, glycan, lipoteichoic acid or combinations thereof.

In an alternative embodiment, the present disclosure provides a method of providing dialysis to a patient. The method comprises providing a dialysis solution, passing the dialysis solution through one or more ion exchange resins so that the substances are removed from the dialysis solution by being adsorbed onto the ion exchange resin. The dialysis solution can then be administered to the patient using any suitable dialysis technique. For example, the dialysis solutions can be used during peritoneal dialysis, such as automated peritoneal dialysis, continuous ambulatory peritoneal dialysis, continuous flow peritoneal dialysis and the like. It should be appreciated that the present disclosure can be used to produce dialysis solutions for a variety of different dialysis therapies to treat kidney failure.

Ready-to-use formulations of dialysis solutions can be prepared in a number of suitable ways. For example, the dialysis solution can comprise first and second dialysis parts that can be separately stored from each other, such as in separate and hydraulically connected chambers of a multi-chamber container, until mixed together to form a mixed solution. In this regard, the ready-to-use formulation can be prepared within a multiple chamber container by mixing its separate dialysis parts within one chamber of the container. This can effectively eliminate the need to manually inject all or at least a portion of the dialysis parts into the container to form the mixed solution, thus ensuring that the ready-to-use formulation can be readily prepared under sterile conditions.

Further, the multiple chamber container can be configured such that one of the dialysis parts can be placed in direct fluid communication with the patient prior to mixing while the other dialysis part cannot be placed in direct fluid communication with the patient prior to mixing. This can provide an added level of safety with respect to the preparation and administration of the ready-to-use formulation of the present disclosure as the single solution that cannot be placed in direct fluid communication with the patient physically cannot be fed to the patient unless it is first mixed with the other component. In this regard, if, by chance, the single solution part that physically cannot be placed in direct fluid communication with the patient were to have an undesirable concentration of constituents, such as potassium, sodium or the like, this configuration would necessarily ensure that the undesirable level of constituents is not fed or administered to the patient.

It should be appreciated that the separate dialysis parts of a multi-part dialysis solution can be housed or contained in any suitable manner such that the individual dialysis parts can be effectively prepared and administered. A variety of containers can be used to house the two parts, such as separate containers (e.g., flasks or bags) that are connected by a suitable fluid communication mechanism. The two or more separate dialysis parts can be separately sterilized and stored.

The dialysis solutions can comprise one or more suitable dialysis components (e.g. ingredients or constituents of a dialysis solution) such as osmotic agents, buffers, electrolytes or combination thereof. A variety of different and suitable acidic and/or basic agents can also be utilized to adjust the pH of the osmotic, buffer and/or electrolyte solutions or concentrates. For example, a variety of inorganic acids and bases can be utilized including hydrochloric acid, sulfuric acid, nitric acid, hydrogen bromide, hydrogen iodide, sodium hydroxide, the like or combination thereof.

Examples of osmotic agents include glucose, fructose, glucose polymers (e.g. maltodextrin, icodextrin, trehalose, cyclodextrins), glucose polymer derivatives (e.g. hydroxyethyl starch, modified starch), polyols, amino acids, peptides, proteins, amino sugars, N-acetyl glucosamine (NAG), glycerol and/or the like and combinations thereof. Examples of the buffers include bicarbonate, lactic acid/lactate, pyruvic acid/pyruvate, acetic acid/acetate, citric acid/citrate, amino acids, peptides, an intermediate of the KREBS cycle and/or the like and combinations thereof.

Examples of electrolytes include calcium, magnesium, sodium, potassium, chloride and/or the like and combinations thereof. For example, the dialysis solutions can comprise one or more electrolytes in the following ranges from: about 100 to about 140 mEq/L of Na⁺, about 70 to about 130 mEq/L of Cl⁻, 0.1 to about 4.0 mEq/L of Ca²⁺, 0.1 to about 4.0 mEq/L of Mg²⁺ and/or 0.1 to about 4.0 mEq/L of K⁺.

The dialysis solutions can preferably contain a dialysis component such as an osmotic agent to maintain the osmotic pressure of the solution greater than the physiological osmotic pressure (e.g. greater than about 285 mOsmol/kg). For example, glucose is the most commonly used osmotic agent because it provides rapid ultrafiltration rates. Other suitable types of osmotic agents can be used in addition to or as a substitute for glucose.

Another family of compounds capable of serving as osmotic agents in peritoneal dialysis solutions is that of glucose polymers or their derivatives, such as icodextrin, maltodextrins, hydroxyethyl starch, and the like. While these compounds are suitable for use as osmotic agents, they can be sensitive to low and high pH, especially during sterilization and long-term storage. Glucose polymers, such as icodextrin, can be used in addition to or in place of glucose in peritoneal dialysis solutions. In general, icodextrin is a polymer of glucose derived from the hydrolysis of corn starch. It has a molecular weight of 12-20,000 Daltons. The majority of glucose molecules in icodextrin are linearly linked with α (1-4) glucosidic bonds (>90%) while a small fraction (<10%) is linked by α (1-6) bonds.

The dialysis solutions or components can also comprise buffering agents such as bicarbonates and acids. The bicarbonates can comprise an alkaline solution such that the bicarbonate can remain stable without the use of a gas barrier overpouch or the like, The individual bicarbonate solution can have a pH that ranges above about 8.6, preferably about 9. The pH of the bicarbonate solution part can be adjusted with any suitable type of ingredient, such as sodium hydroxide and/or the like. Illustrative examples of the bicarbonate solution of the present disclosure can be found in U.S. Pat. No. 6,309,673, entitled BICARBONATE-BASED SOLUTION IN TWO PARTS FOR PERITONEAL DIALYSIS OR SUBSTITUTION IN CONTINUOUS RENAL REPLACEMENT THERAPY, issued on Oct. 30, 2001, the disclosure of which is herein incorporated by reference.

The acids can comprise one or more physiological acceptable acids, such as lactic acid, pyruvic acid, acetic acid, citric acid, hydrochloric acid and the like. The acids can be in an individual solution having a pH that ranges from about 5 or less, about 4 or less, about 3 or less, about 2 or less, about 1 or less, and any other suitable acidic pH. The use of an organic acid, such as lactic acid, alone or in combination with another suitable acid, such as a suitable inorganic acid including hydrochloric acid, another suitable organic acid (e.g. lactic acid/lactate, pyruvic acid/pyruvate, acetic acid/acetate, citric acid/citrate) and the like in the acid solution can make the solution more physiologically tolerable.

As discussed previously, the dialysis solutions of the present disclosure can be used in a variety of suitable applications. For example, the dialysis solutions can be used during peritoneal dialysis, such as automated peritoneal dialysis, continuous ambulatory peritoneal dialysis, continuous flow peritoneal dialysis and the like. It should be appreciated that the present disclosure can be used in a variety of different and suitable dialysis therapies to treat kidney failure.

Although the present disclosure, in an embodiment, can be utilized in methods providing a dialysis therapy for patients having chronic kidney failure or disease, it should be appreciated that the present disclosure can be used for acute dialysis needs, for example, in an emergency room setting. Lastly, as one of skill in the art appreciates, the intermittent forms of dialysis therapy may be used in the in-center, self/limited care as well as the home settings.

### EXAMPLES

By way of example and not limitation, the following examples are illustrative of various embodiments of the present disclosure.

The purpose of these experiments was to demonstrate removal and isolation of the substance(s) in an implicated EXTRANEAL® dialysis solution that was responsible for positive responses in a Peripheral Blood Mononuclear Cell (PBMC) IL-6 Release Assay (the “PBMC IL-6 assay”). By performing ion capture chromatography with a DEAE SEPHAROSE® anion exchange resin, a method for separating the IL-6 inducing substance (the “IL-6 IS”) from the implicated dialysis solution comprising icodextrin was discovered.

### Test Articles

The following experiments used two or more of the following solutions to demonstrate the utility of employing ion exchange methods to remove inflammatory substances from a dialysis solution:
1) EXTRANEAL® dialysis solution, Lot #07G06G40 (control lot, denoted “A”)
2) EXTRANEAL® dialysis solution, Lot #06K23G38 (implicated lot, denoted “B”)
3) DIANEAL® dialysis solution, Baxter cat #5B5203

### Experiment #1—DEAE SEPHAROSE® Anion Ion Exchange Resin—Run #1

### Materials:

DEAE SEPHAROSE® fast flow, Amersham Biosciences

Sodium Phosphate Monobasic, Mallinckrodt

Sodium Chloride, Mallinckrodt

Ethanol, Spectrum

Sterile Water for Irrigation, Baxter

0.9% Sodium Chloride for Irrigation (Saline), Baxter

Three columns of approximately 20 ml bed volume were poured and prepared for sample by first running 160 ml of “sterile” 20% ethanol over each column, followed by 100 ml of sterile saline. The columns were equilibrated with 0.01M sodium phosphate at pH 7.2. After the columns were equilibrated, 200 ml of each sample was loaded onto one of the columns and washed until the background absorbance levels (A260 nm) were obtained. The columns were then eluted with PBS containing 1.5 M sodium chloride. Fractions were collected periodically during the loading, wash and elution phases of the runs. The absorbance of these fractions at 260 was also monitored to assure that the wash and elution steps were complete.

All three columns were treated identically with the exception of the sample. The sample for the first column was A (200 mL), the second column was B (200 mL) and the sample for the third column was the Dianeal sample (200 mL). Selected fractions from all three columns were submitted for IL-6 analysis.

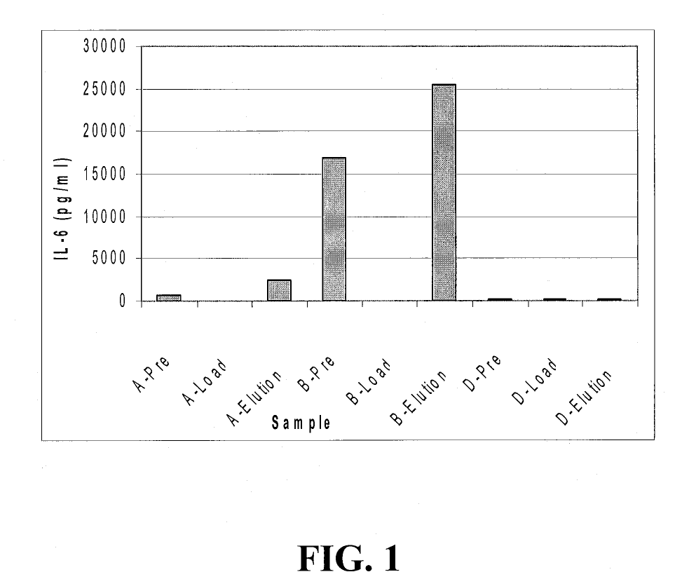

The IL-6 results are shown in FIG. 1. From this run, it was clear that the IL-6 response from the sample (see Sample B in FIG. 1) was completely depleted from the load fraction (B-Load), and was observed to be high in the elution fractions (B-Elution). There was low activity in the EXTRANEAL® dialysis solution control (A Fractions) and no activity in the DIANEAL® dialysis solution control (D Fractions). Thus, the inflammatory (IL-6 IS) material in the EXTRANEAL® dialysis solution was effectively removed by passing the solution over this ion exchange matrix.

### Experiment #2—DEAE SEPHAROSE® Anion Ion Exchange Resin—Run #2

Run #2 was a repeat of Run #1 (using the columns with approximately 20 mL bed volumes) comparing the two lots of EXTRANEAL® dialysis solution but this time loading was done with 400 mL of sample instead of 200 mL. The columns were washed with 250 mL of 0.01 M phosphate buffer at pH 7.2. After washing was completed, the columns were eluted with approximately 90 mL of PBS containing 1.5M sodium chloride. Fractions were collected during the sample loading, wash and elution phases of the run, and were separately pooled (i.e. all A fractions were pooled together and all B fractions together to produce the respective Load, Wash and Elution pools) and concentrated to 0.5 mL in an Amicon Ultra-4 Ultracel with a 5000 molecular weight cut-off (MWCO) membrane (Millipore). All of the various pooled fractions were washed in the Ultra-4 Ultracel devices (3 times) resulting in a final volume of 0.5 mL. The final pools were submitted for total icodextrin analysis and IL-6 analysis.

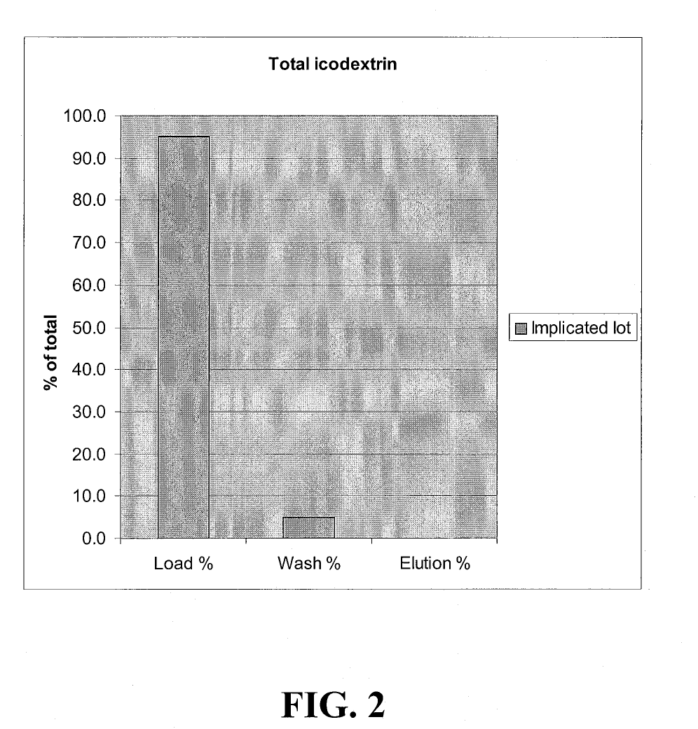

Based on past elution results, the pooled fractions were desalted before being subjected to IL-6 testing. The pooled, desalted fraction from the contaminated EXTRANEAL® dialysis solution (solution B) gave a high IL-6 response in this assay. This material, along with load and wash fractions, were submitted for icodextrin quantification. The mass balance results are given in Table 1 and FIG. 2. This showed that all of the icodextrin went through the column when the sample was loaded with a small percentage coming off when the column was washed, and no icodextrin was detectable in the elution fractions. Thus, this method successfully separated the IL-6 IS from the icodextrin.

TABLE 1
Mass balance results for icodextrin in DEAE Run #3

| | Dialysis Solution | Dialysis Solution |
|---|---|---|
| | Lot # 07G06G40 | Lot # 06K23G38 |
| Starting quantity (mg) | 30000 | 30000 |
| Load quantity (mg) | 28831 | 27409 |
| Load % | 94.8% | 95.2% |
| Wash quantity (mg) | 1572 | 1379.4 |
| Wash % | 5.2% | 4.8% |
| Elution quantity (mg) | BDL | BDL |
| Elution % | 0.0% | 0.0% |
| Total quantity (mg) | 30407 | 28788 |
| Total recovery % | 101.4% | 96.0% |

BDL = below detectable limits

### Experiment #3—DEAE SEPHAROSE® Anion Ion Exchange Resin—Run #3

Run #3 was a scale up of Run #1. Larger columns with bed volumes of approximately 100 mL were poured. The columns were prepared for “sterile” run with 20% ethanol and saline washes as previously done. They were then pretreated running 250 mL of phosphate buffer saline (PBS) containing 1.5M sodium chloride over each column to pre-strip each column before use. The columns were then equilibrated in 0.01 M phosphate buffer pH 7.2. After equilibration was complete, 2 L of sample was loaded on each of the corresponding columns overnight. The columns were then washed with the equilibration buffer. The last 5 mL of each wash was collected and set aside. The columns were eluted with 1× PBS containing 1.5M sodium chloride. Fractions were collected periodically through the sample loading, wash and elution steps. Selected samples were submitted for IL-6 analysis.

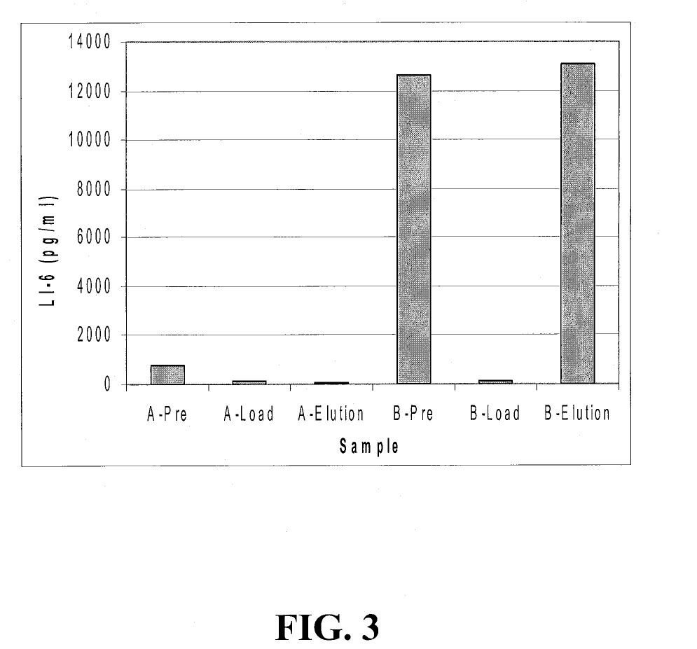

The Il -6 results from the scaled up run using larger column bed volumes and 2000 mL of EXTRANEAL® dialysis solution are shown in FIG. 3. IL-6 testing of fractions indicated that the scale up was successful, with again the IL-6 IS observed only in the elution fractions from Solution B demonstrating that the DEAE column successfully removed the IL-6 IS from the contaminated dialysis solution.

### Conclusion

A method for separating the IL-6 IS from icodextrin was discovered using ion capture chromatography with a DEAE SEPHAROSE® anion exchange resin. Passing EXTRANEAL® dialysis solution over the DEAE SEPHAROSE® column resulted in removal of the IL-6 inducing activity from the run-through EXTRANEAL® dialysis solution fraction containing all of the icodextrin. The IL-6 inducing substance was subsequently eluted off of the column with a high salt wash.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for removing a substance from a peritoneal dialysis solution, the method comprising:
providing a peritoneal dialysis solution; and
passing the peritoneal dialysis solution through an ion exchange resin so that at least a portion of the overall amount of the substance is removed from the peritoneal dialysis solution by being adsorbed onto the ion exchange resin.

2. The method of claim 1 further comprising spray drying the dialysis solution to form a spray dried dialysis ingredient after the dialysis solution has passed through the ion exchange resin.

3. The method of claim 2 further comprising packaging the spray dried dialysis ingredient into a sterile container.

4. The method of claim 1 further comprising packaging the dialysis solution into a sterile container after the dialysis solution has passed through the ion exchange resin.

5. The method of claim 1 further comprising passing a basic solution through the ion exchange resin prior to passing the dialysis solution through the ion exchange resin.

6. The method of claim 5, wherein the basic solution comprises a pH greater than 7.0.

7. The method of claim 1, wherein the ion exchange resin comprises an anion exchange resin.

8. The method of claim 1, wherein the dialysis solution comprises a glucose polymer as an osmotic agent.

9. The method of claim 8, wherein the glucose polymer is icodextrin.

10. The method of claim 1, wherein the substance is an IL-6 inducing substance.

11. A method for removing a substance from a dialysis component, the method comprising:
dissolving the dialysis component in a solution; and
passing the dissolved dialysis component solution through an ion exchange resin so that at least a portion of the overall amount of the substance is removed from the dialysis component solution by being adsorbed onto the ion exchange resin.

12. The method of claim 11 further comprising spray drying the dialysis component solution to form a spray dried dialysis ingredient after the dialysis component solution has passed through the ion exchange resin.

13. The method of claim 12 further comprising preparing a peritoneal dialysis solution using the spray dried dialysis ingredient.

14. The method of claim 11, wherein the dialysis component is an osmotic agent for a dialysis solution.

15. A method for manufacturing a peritoneal dialysis solution, the method comprising:
providing at least one peritoneal dialysis component;
dissolving the peritoneal dialysis component in a solution;
passing the peritoneal dialysis component solution through an ion exchange resin so that at least a portion of the overall amount of the charged contaminant is removed from the solution by being adsorbed onto the ion exchange resin; and
preparing a peritoneal dialysis solution using the peritoneal dialysis component.

16. The method of claim 15, wherein at least one of the peritoneal dialysis components is icodextrin.

17. A method for removing microbial contaminants in a peritoneal dialysis solution, the method comprising:
providing a peritoneal dialysis solution; and
passing the peritoneal dialysis solution through an anion exchange resin so that at least a portion of the overall amount of the microbial contaminants are removed from the peritoneal dialysis solution by being adsorbed onto the ion exchange resin.

18. The method of claim 17 further comprising spray drying the dialysis solution to form a spray dried dialysis ingredient after the dialysis solution has passed through the ion exchange resin.

19. The method of claim 18 further comprising packaging the spray dried dialysis ingredient into a sterile container.

20. The method of claim 17 further comprising packaging the dialysis solution into a sterile container after the dialysis solution has passed through the ion exchange resin.

21. The method of claim 17 further comprising passing a basic solution through the ion exchange resin prior to passing the dialysis solution through the ion exchange resin.

22. The method of claim 21, wherein the basic solution comprises a pH greater than 7.0.

23. The method of claim 17, wherein the dialysis solution comprises a glucose polymer as an osmotic agent.

24. The method of claim 23, wherein the glucose polymer is icodextrin.

25. The method of claim 17, wherein the microbial contaminants are selected from the group consisting of peptide, glycan, lipoteichoic acid and combinations thereof.

26. A method of providing dialysis to a patient, the method comprising:
providing a dialysis solution;
passing the dialysis solution through an ion exchange resin so that the substances are removed from the dialysis solution by being adsorbed onto the ion exchange resin; and
administering the dialysis solution to the patient.
